(19) United States
(12) Patent Application Publication
(10) Pub. No.: US 2016/0128858 A1
(43) Pub. Date: May 12, 2016

(54) Shoe Interfaced Ankle Support Apparatus

(71) Applicant: Tyrone Wilson, Washington, NC (US)

(72) Inventor: Tyrone Wilson, Washington, NC (US)

(21) Appl. No.: 14/966,687

(22) Filed: Dec. 11, 2015

Related U.S. Application Data
(63) Continuation-in-part of application No. 14/198,782, filed on Mar. 6, 2014.

(51) Int. Cl. A61F 5/01; A61F 5/04

(52) U.S. Cl. CPC A61F 5/012; A61F 5/0127; A61F 5/0111; A61F 5/04

(57) ABSTRACT

A shoe-interfaced ankle support apparatus includes a leg support member wearable on a user's lower leg. A primary foot sling is coupled to the side wall of the leg support portion adjacent an open bottom that is configured to extend loosely across the open bottom of the leg support portion so as to selectively cradle the foot of the user while in a shoe. A shoe interface member includes a proximal end coupled to the side wall of the leg support portion that extends away, the shoe interface member having a plurality of apertures configured to register with respective holes of the shoe's lace portion and to receive the shoelace of the shoe therethrough. The leg support member may include an inflatable bladder movable between inflated and deflated configurations for supporting a user's ankle. Heat and nerve stimulation sleeves may also be situated in the leg support member.

### REFERENCE TO RELATED APPLICATION

This is a Continuation-in-Part and claims the benefit of application Ser. No. 14/198,782 filed on Mar. 6, 2014 titled “Shoe Interfaced Ankle Support Apparatus,” now pending and which is incorporated completely herein by reference.

### BACKGROUND OF THE INVENTION

This invention relates generally to ankle braces and, more particularly, to an ankle and leg support apparatus that is worn on the lower leg of a person and which interfaces with the shoe lace portion and shoe laces of any lace-type shoe.

A sprained ankle is a common injury to the ligaments in the ankle region of a persons lower leg and foot. Sometime called a sprain, a twisted ankle, a rolled ankle, or the like, the condition is caused by the partial tearing of ligaments of the ankle. A sprained ankle is usually caused when a person's foot “rolls” or collapses due to an awkward step or when an uneven ground surface urges the foot to essentially fold over when weight is applied over the foot while walking or running.

Various devices and treatments have been proposed in the art for enhancing healing of an ankle sprain or simply for minimizing the pain of the injury. For instance, elastic wraps and braces are beneficial in that they provide a compression force that gives a person reassurance while walking. Of course, crutches may be used by a person with a sprained ankle so as to eliminate any weight-bearing on the affected ankle. Although assumably effective for their intended purposes, the existing devices and proposals do not provide an apparatus that provides compression to the lower leg, sling cradling of a person's foot to lessen its weight bearing, and the ability to tighten the compression by tightening the laces of a shoe to which the apparatus is interfaced.

Therefore, it would be desirable to have a shoe-interfaced ankle support apparatus that may be worn about the lower leg of a person with a sprained ankle and which cradles the person's foot inside a shoe. Further, it would be desirable to have a shoe-interfaced ankle support apparatus that is coupled to the lace portion of a lace-up type shoe and can be tightened with the shoe laces. In addition, it would be desirable to have a shoe-interfaced ankle support apparatus that includes an inflatable air bladder for additional support, a heating sleeve and a transcutaneous electrical nerve stimulation (“TENS”) sleeve for therapeutic treatment of a user's lower leg.

### SUMMARY OF THE INVENTION

A shoe-interfaced ankle support apparatus according to the present invention includes a leg support member having a continuous side wall defining an open top and open bottom configured to be worn on the user's lower leg. The support apparatus includes a primary foot sling having opposed first and second ends coupled to the side wall of the leg support portion adjacent the closed bottom, the foot sling having a length configured to extend loosely across the open bottom of the leg support portion so as to selectively receive and cradle the foot of the user. A shoe interface member includes a proximal end operatively coupled to the side wall of the leg support portion adjacent the open bottom and extending away from the side wall, the shoe interface member having a plurality of spaced apart apertures configured to register with respective holes of the lace portion of the shoe and to receive the shoelace of the shoe therethrough.

In another aspect, an air bladder is in operative communication with an inner surface of the side wall of the leg support member, the air bladder being movable between an inflated configuration and a deflated configuration, preferably with a manual pump. A heat sleeve is operatively coupled to the inner surface of the leg support member, the heat sleeve having a heating element situated internally and configured to warm the leg support member when energized. Further, a transcutaneous electrical nerve stimulation (“TENS”) sleeve operatively coupled to the inner surface of the leg support member, the TENS sleeve configured to therapeutically stimulate nerves in a user's leg when wearing the leg support member and when energized. The leg support member may include at least one pouch configured to a hot pack or cold pack.

Therefore, a general object of this invention is to provide an ankle support apparatus that is worn about the foot and lower leg of a person and that interfaces with the shoe laces of any lace-type shoe.

Another object of this invention is to provide an ankle support apparatus, as aforesaid, that can be increasingly tightened about a person's ankle by lacing the shoe tighter.

Still another object of this invention is to provide an ankle support apparatus, as aforesaid, that can be increasingly tightened about a person's lower leg by tightening the leg support member.

Yet another object of this invention is to provide an ankle support apparatus, as aforesaid, that cradles a person foot and ankle in a foot sling when the apparatus is worn and the apparatus is interfaced with a shoe.

A further object of this invention is to provide an ankle support apparatus, as aforesaid, that is easy for a user to wear and to lace to a regular lace-type shoe.

A still further object of this invention is to provide an ankle support apparatus, as aforesaid, having an inflatable air bladder situated in the leg support member for selectively increasing the tightened support of the ankle support apparatus on a user's ankle.

Another object of this invention is to provide an ankle support apparatus, as aforesaid, in which the leg support member includes a heating sleeve configured to impart heat to the leg support member when energized.

Still another object of this invention is to provide an ankle support apparatus, as aforesaid, in which the leg support member includes a transcutaneous electrical nerve stimulation (“TENS”) sleeve that is configured to therapeutically stimulate nerves in a user's leg when wearing said leg support member.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1a is a perspective view of a shoe-interfaced ankle support apparatus according to a preferred embodiment of the present invention with a sleeve fastener in a closed configuration;

FIG. 1b is an isolated view on an enlarged scale taken from FIG. 1a;

FIG. 1c is an isolated view on an enlarged scale taken from FIG. 1a;

FIG. 1d is a perspective view from another angle of the ankle support apparatus as in FIG. 1a with the sleeve fastener in a released configuration;

FIG. 1e is an isolated view on an enlarged scale taken from FIG. 1d;

FIG. 2a is a perspective view of a shoe-interfaced ankle support apparatus according to another embodiment of the present invention with a primary pocket fastener in a closed configuration;

FIG. 2b is an isolated view on an enlarged scale taken from FIG. 2a;

FIG. 2c is a perspective view of a padded panel;

FIG. 2d is an isolated view on an enlarged scale taken from FIG. 2a;

FIG. 2e is an isolated view on an enlarged scale taken from FIG. 2a;

FIG. 2f is an isolated view on an enlarged scale taken from FIG. 2a;

FIG. 2g is an isolated view on an enlarged scale taken from FIG. 2a;

FIG. 2h is a perspective view from another angle as in FIG. 2a;

FIG. 3a is a perspective view of the ankle support apparatus as in FIG. 2a from another angle illustrated with a secondary pocket fastener in an open configuration so as to receive a padded panel;

FIG. 4a is a perspective view of a shoe-interfaced ankle support apparatus according to another embodiment of the present invention

FIG. 4b is an isolated view on an enlarged scale taken from FIG. 4a;

FIG. 4c is an exploded view of the ankle support apparatus as in FIG. 4a;

FIG. 4d is an isolated view on an enlarged scale taken from FIG. 4a;

FIG. 4e is an isolated view on an enlarged scale taken from FIG. 4a;

FIG. 5 is a perspective view of a shoe-interfaced ankle support apparatus according to another embodiment of the present invention;

FIG. 6 is a perspective view from another angle of the support apparatus as in FIG. 5;

FIG. 7 is a side view of the support apparatus as in FIG. 5;

FIG. 8a is a sectional view taken along line 8a-8a of FIG. 7;

FIG. 8b is an isolated view on an enlarged scale taken from FIG. 8a;

FIG. 9 is an exploded view of the support apparatus as in FIG. 5;

FIG. 10 is a perspective view from an underneath angle of the support apparatus as in FIG. 5;

FIG. 11 is an isolated view on an enlarged scale taken from FIG. 10;

FIG. 12a is a perspective view of a front portion of a leg support member according to the present invention;

FIG. 12b is a perspective view from another angle of the front portion of the leg support member of FIG. 12a;

FIG. 13 is a perspective view of a heat sleeve removed from the leg support member shown in FIG. 5;

FIG. 14a is a perspective view of a manual pump for inflating the air bladder as in FIG. 6;

FIG. 14b is a side view of the pump shown in FIG. 14a;

FIG. 15 is an isolated view of a remote control device related to the transcutaneous electrical nerve stimulation (“TENS”) sleeve shown in FIG. 6;

FIG. 16 is an isolated view of a controller related to the heat pad shown in FIG. 6;

FIG. 17 is a perspective view of a transcutaneous electrical nerve stimulation (“TENS”) sleeve illustrated in FIG. 9;

FIG. 18 is an elevated perspective view of the support apparatus illustrated in FIG. 5;

FIG. 19a is a side view of the support apparatus as in FIG. 18; and

FIG. 19b is a sectional view taken along line 19b-19b of FIG. 19a.

### DESCRIPTION OF THE PREFERRED EMBODIMENT

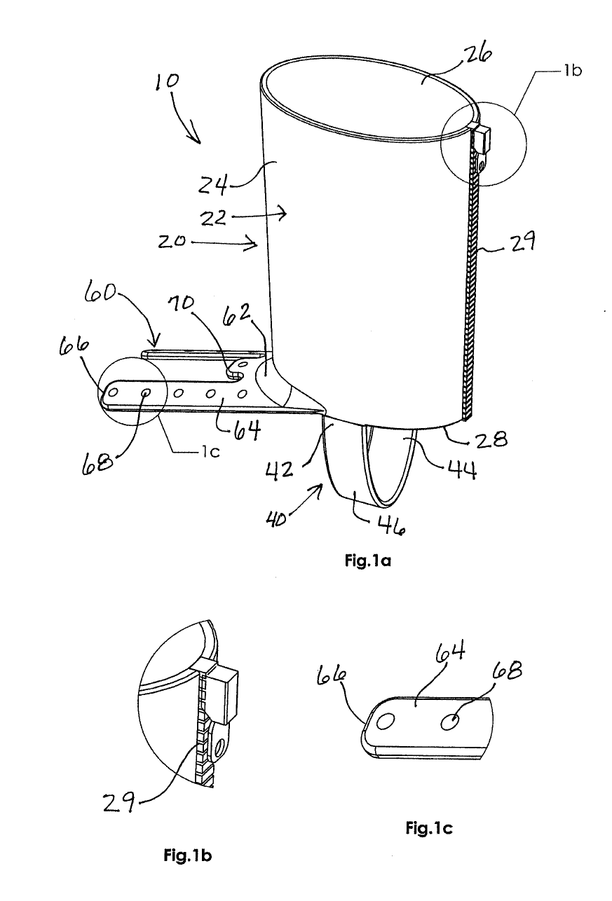

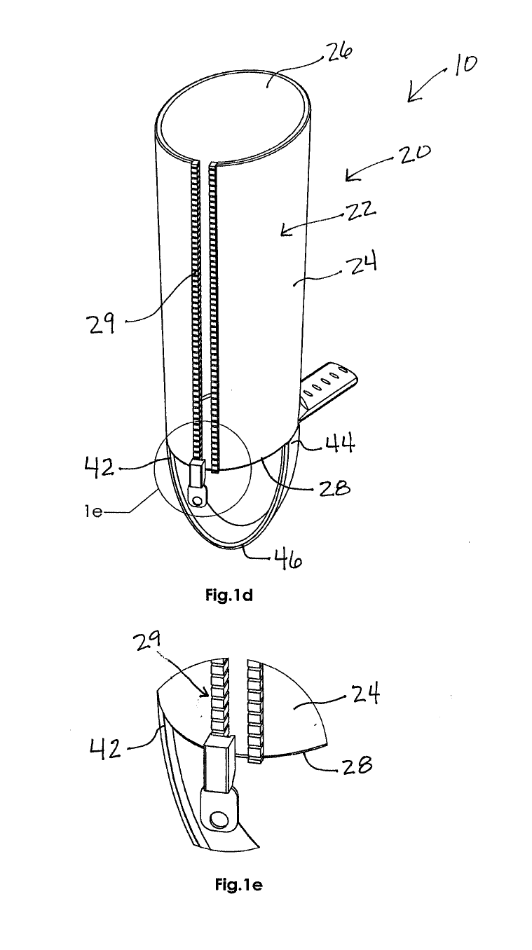

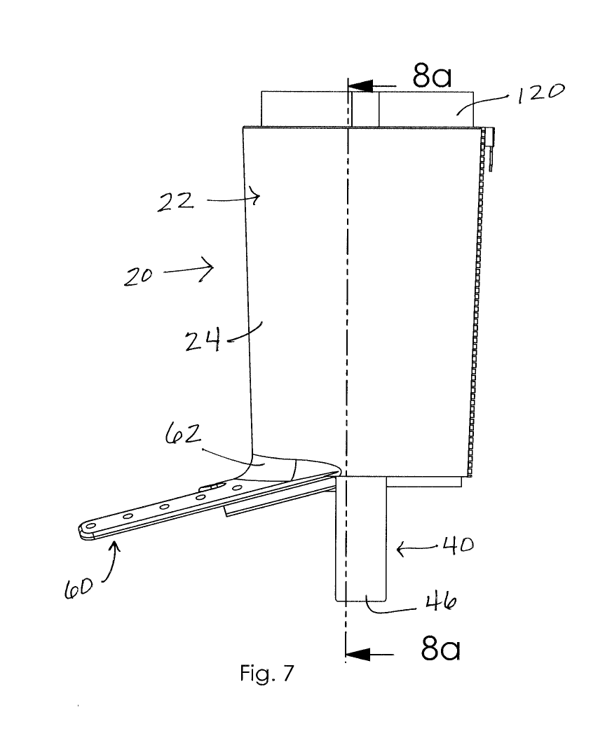

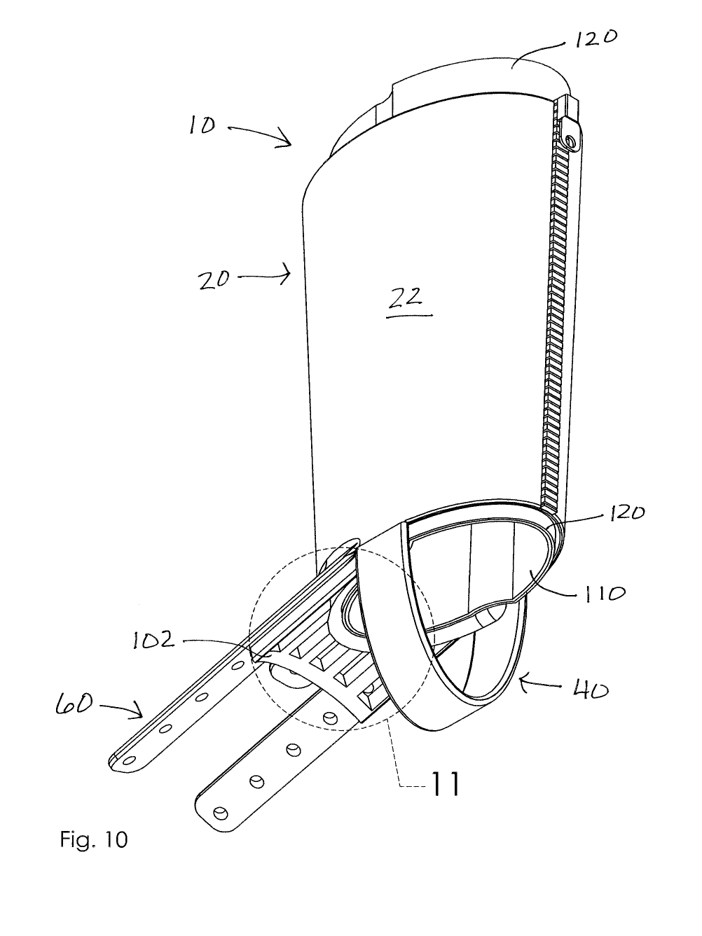

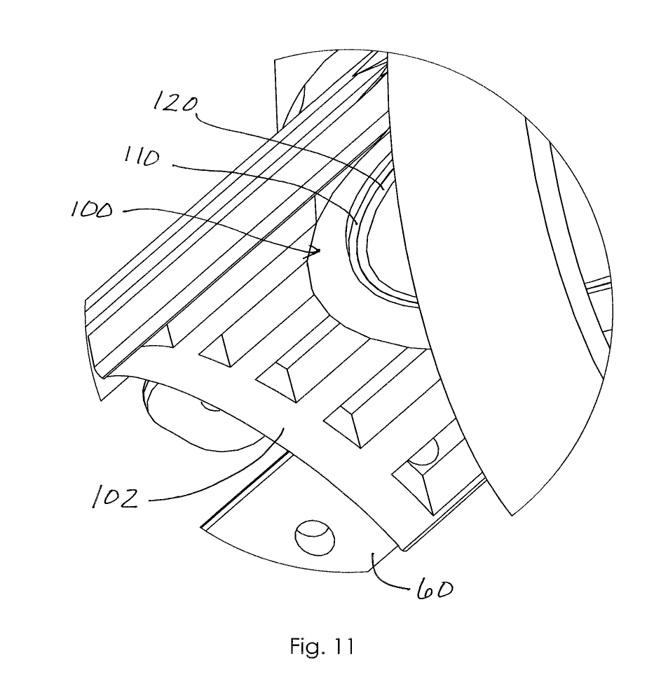

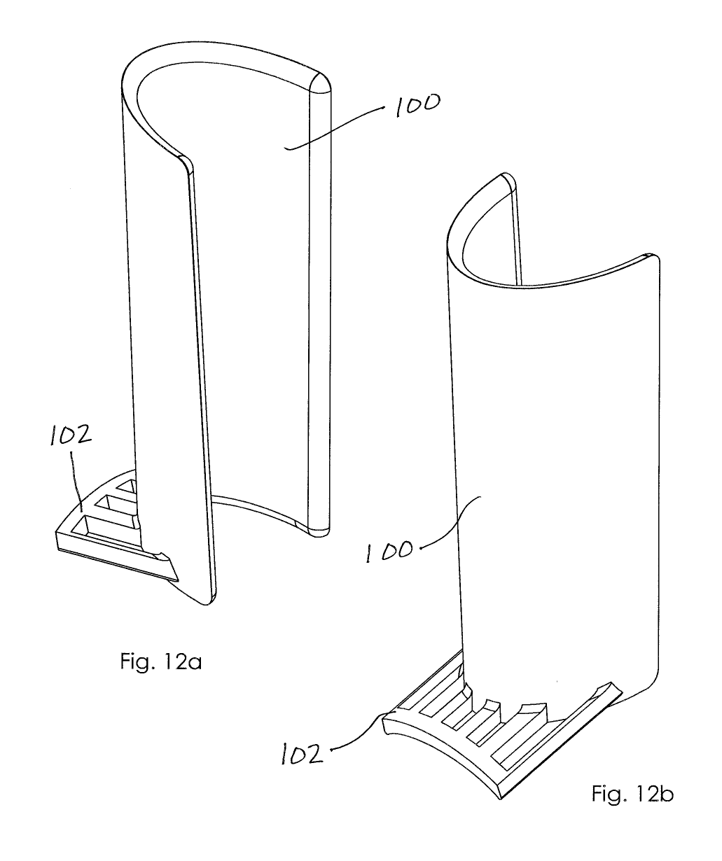

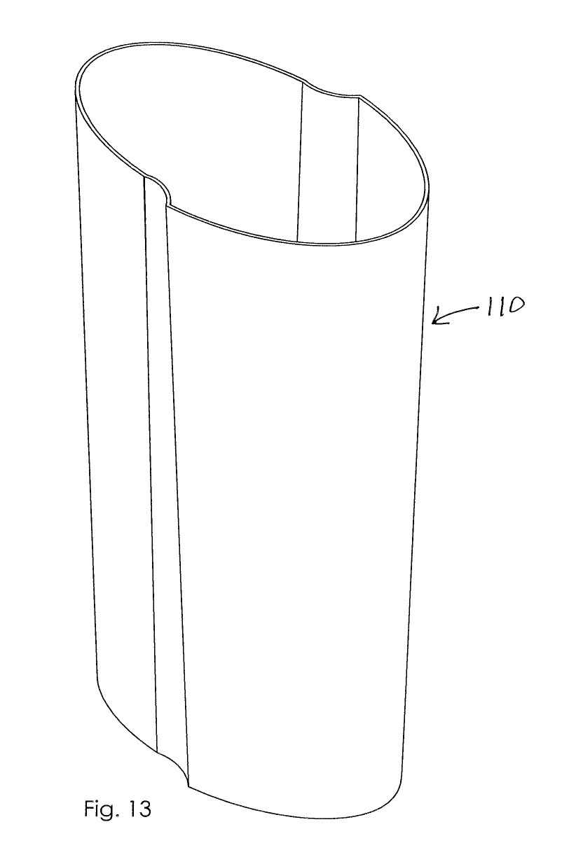

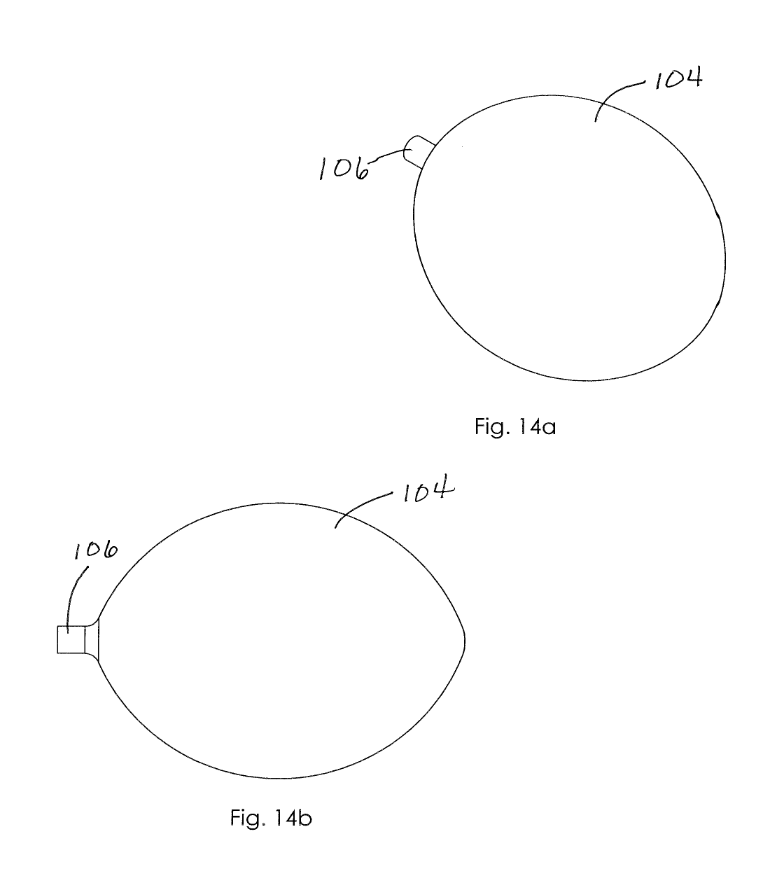

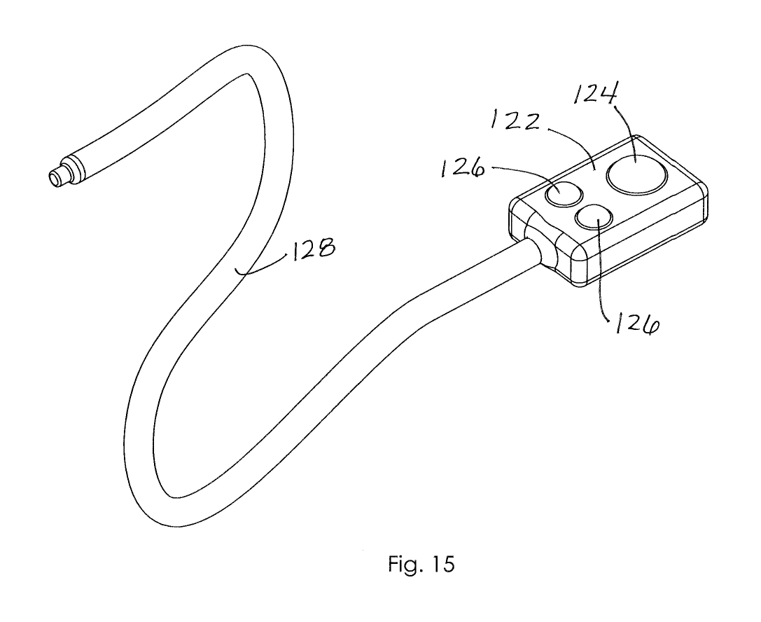

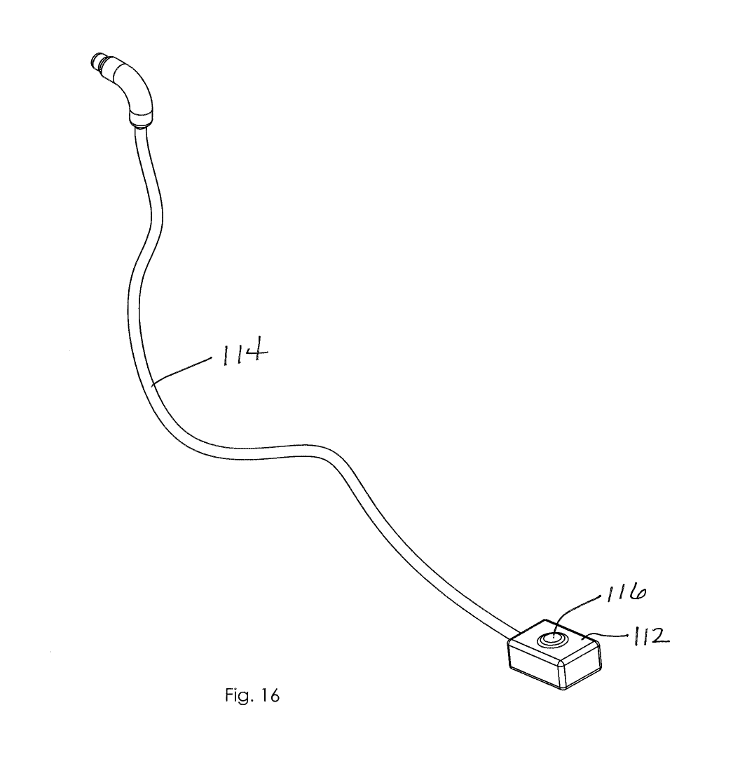

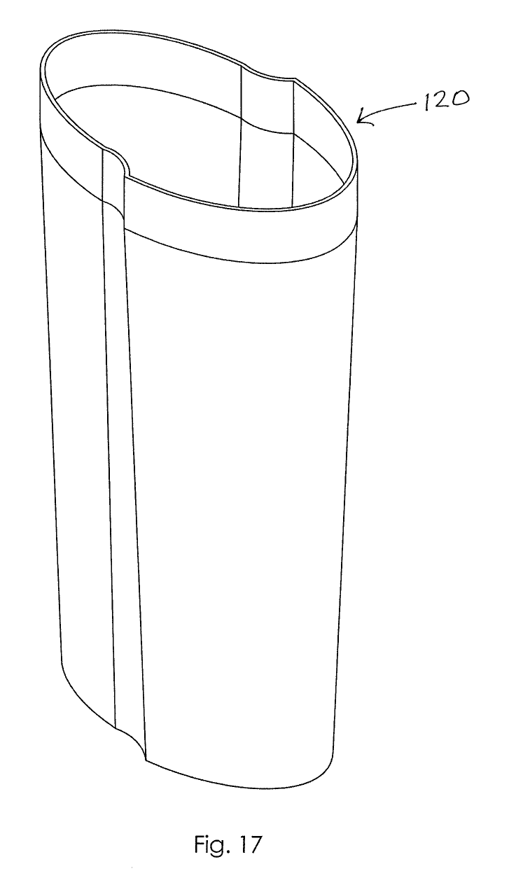

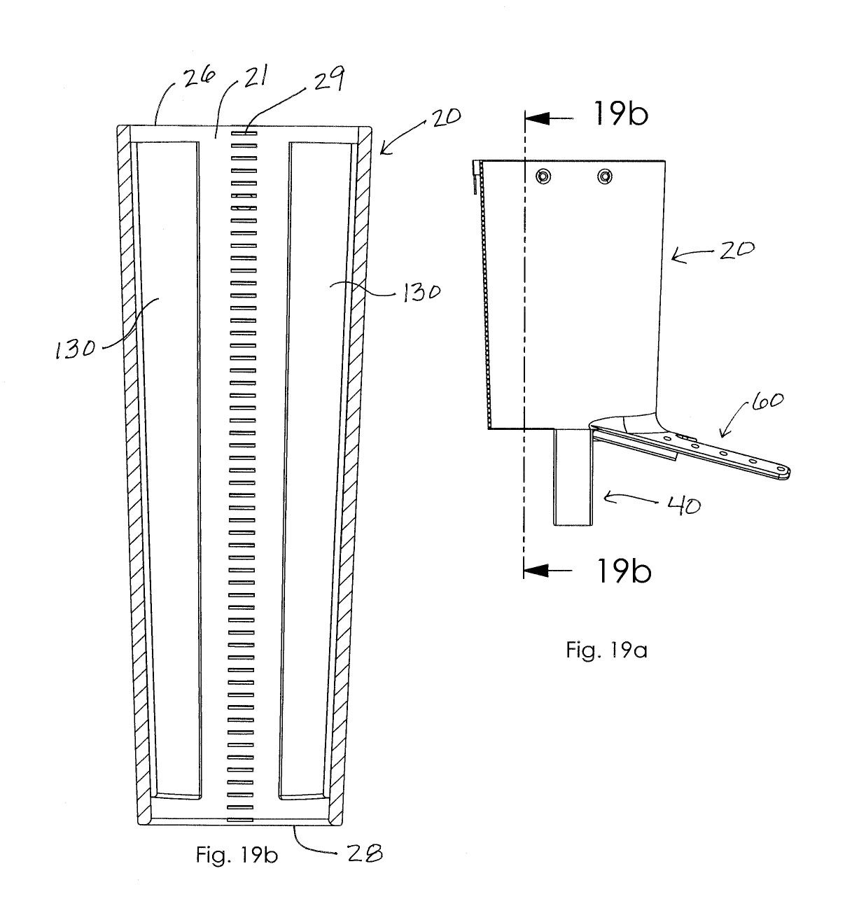

A shoe-interfaced ankle support apparatus according to a preferred embodiment of the present invention will now be described with reference to FIGS. 1a to 19b. The ankle support apparatus 10 includes a leg support member 20, a primary foot sling 40, and a shoe interface member 60.

The leg support member 20 may include a sleeve 22 having a continuous side wall 24 that defines an open top 26, open bottom 28, and open interior space between the ends (FIG. 1a). In one embodiment, the sleeve 22 may be constructed of an elastic material and is configured to be worn on the lower leg of a person seeking relief from a sprained ankle. As with traditional ankle or leg support products, the sleeve 22 may be pulled on over a person's foot and pulled upwardly to the lower leg (e.g. calf) area. The sleeve 22 is configured to provide compression to an affected area and give reassurance of support to the wearer.

In one embodiment, the sleeve 22 may include first and second edges that are normally separated from one another so as to define an open side (FIG. 1d). The first and second edges, however, may be selectively coupled together with a sleeve fastener 29 such as a zipper (FIG. 1a). Specifically, the sleeve 22 is movable between a closed configuration (FIG. 1a) when the sleeve fastener 29 is fully engaged and an open configuration (FIG. 1d) when the sleeve fastener 29 is fully released. In use, the sleeve fastener 29 may be released and the sleeve side wall 24 wrapped around a person's lower leg and then the sleeve fastener 29 reengaged. Although the sleeve fastener 29 is preferably a zipper, the sleeve fastener 29 may also include a hook and loop combination, clasp, snap combination, or other suitable fastener.

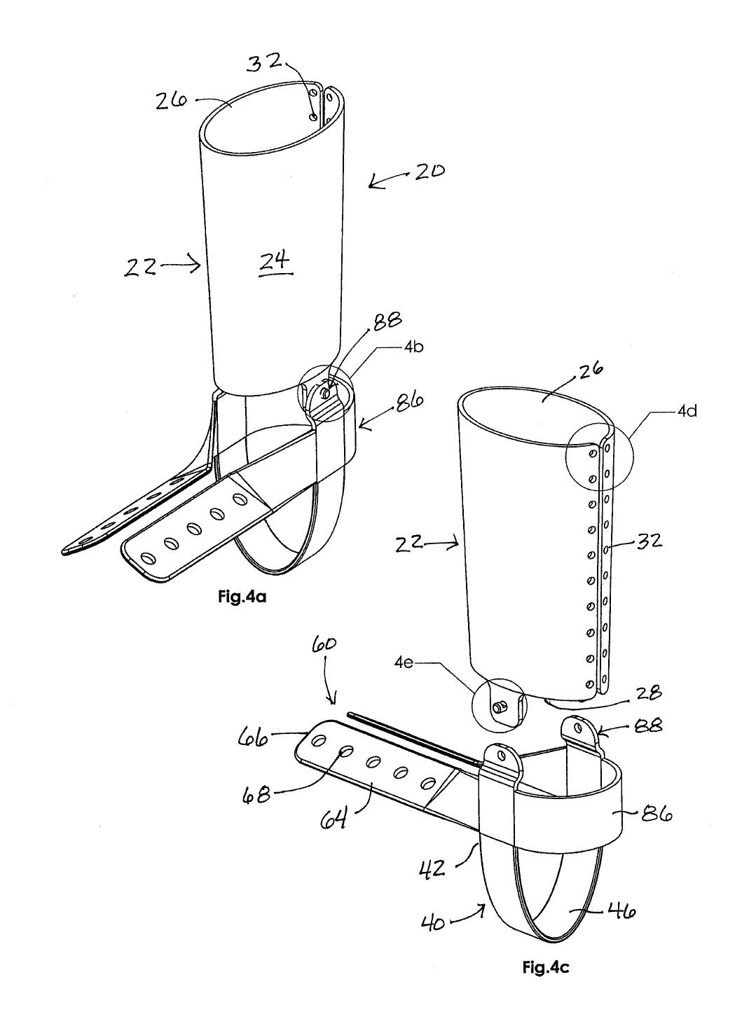

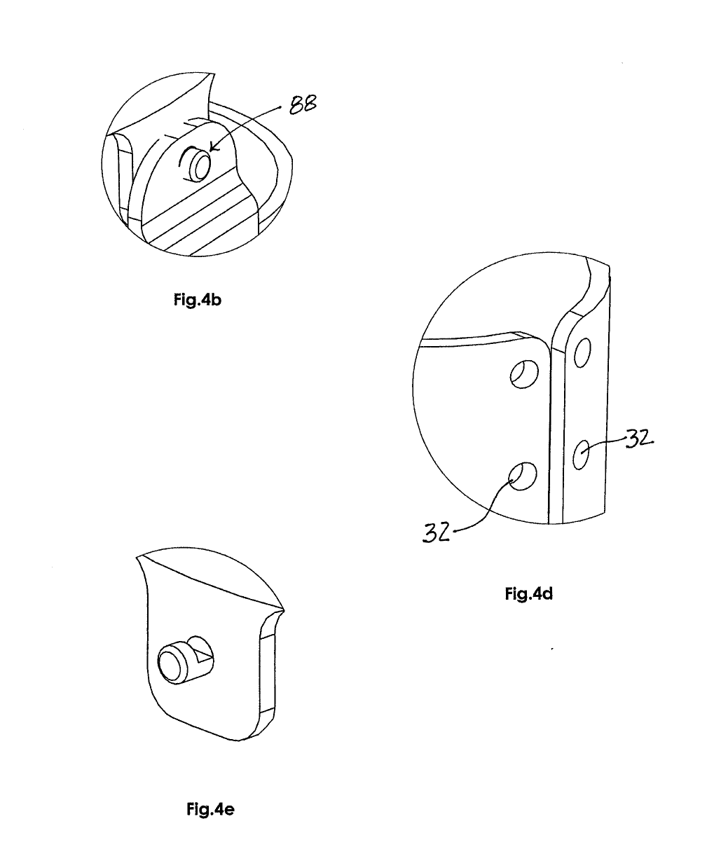

In one embodiment, the sleeve fastener 29 may include a lacing arrangement. More particularly, the side wall 24 of the sleeve 22 adjacent the first and second side edges may define a plurality of lace holes 32 spaced apart between the open top 26 and open bottom 28 thereof (FIGS. 4a and 4c). A shoe lace (not shown) may then laced through respective holes and tightened as desired by the user in a manner similar to lacing up a boot, roller skate, or the like. In this manner, the leg support member 20 may be tightened about a user's leg to a desired tightness and compression.

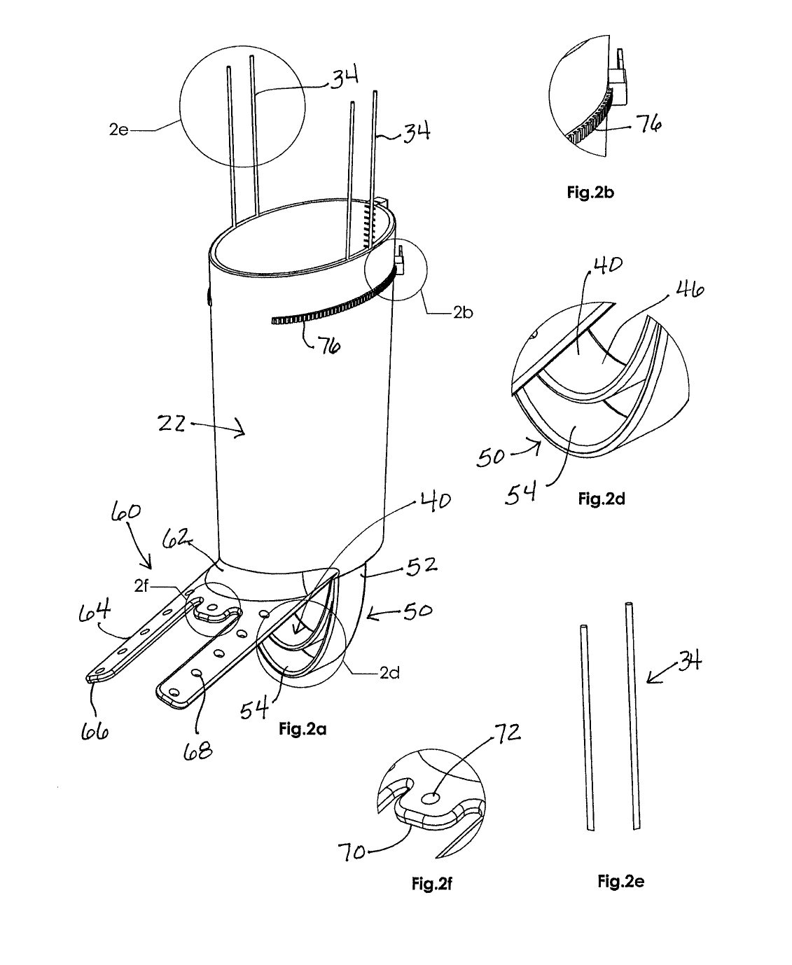

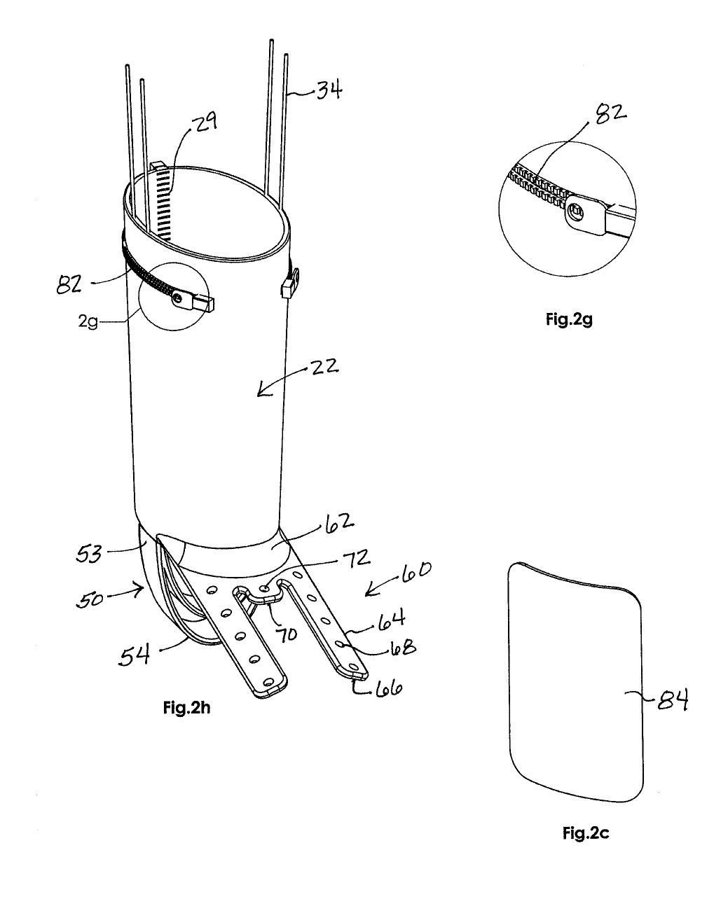

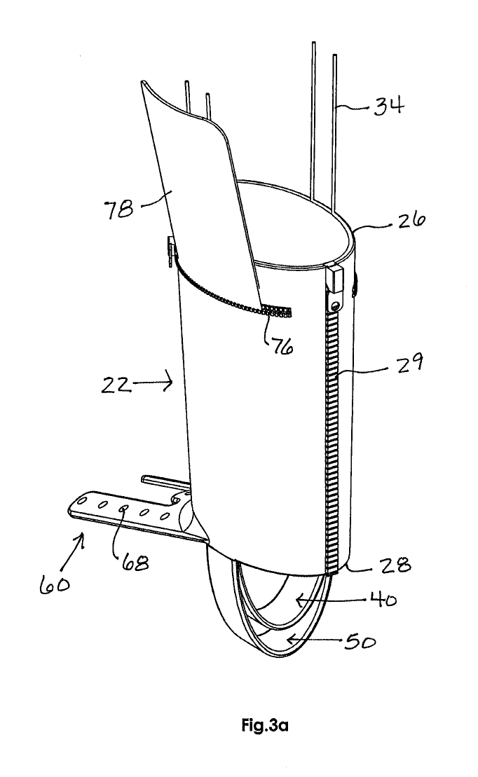

In another embodiment, the sleeve 22 may be tightened using an internal network of cords 34. More particularly, the sleeve fastener 29 includes at least a pair of cords 34 embedded in a network arrangement within an interior area of the sleeve 22 and having free ends that extend upwardly from the sleeve 22 adjacent the open top 26 thereof (FIG. 2a). For instance, the cords 34 may be situated in a generally spiral arrangement throughout the side wall 24 of the sleeve 22 so as to tighten the sleeve 22 when worn on a user's leg and the cords 34 are pulled upwardly.

The primary foot sling 40 may include opposed first 42 and second 44 ends operatively coupled to opposed points of the side wall 24 of the leg support member 20, respectively, adjacent the open bottom 28. In one embodiment, the foot sling ends may be coupled directly and permanently to the sleeve 22 (FIG. 1a). In another embodiment, the ends of the primary foot sling 40 may be coupled to an ankle/leg interface 88 that is situated intermediate the primary foot sling 40 and sleeve 22, as will be described in more detail later (FIG. 4a).

The primary foot sling 40 includes a length and configuration so as to extend loosely between the opposed points of attachment to the side wall 24 of the leg support member 20. In other words, the primary foot sling 40 includes a generally U-shaped cradle portion 46 that hangs downwardly from the leg support member 20 and into a user's shoe so as to cradle a foot. The primary foot sling 40 may be constructed of an elastic material that is configured to receive and cradle a heel portion of a user's foot. In use, the leg support member 20 may be securely worn about the lower leg portion of a user's leg so that the primary foot sling 40 forms a cradle for the user's foot. When the user puts on a shoe, the cradle is configured to remove or relieve the normal weight or force of the foot upon the sole of the shoe.

In another embodiment, a secondary foot sling 50 is situated below the primary foot sling 40 and configured to enhance the support of a user's foot (FIG. 2a). More particularly, the secondary foot sling 50 includes opposed first 52 and second 53 ends coupled to opposed points of the side wall 24 of the leg support member 20, the secondary foot sling 50 having a secondary cradle portion 54 configured to extend loosely across the open bottom 28 thereof in a cradle configuration manner substantially similar to the primary foot sling 40 described above. In one embodiment, the secondary foot sling 50 may have a less flexible construction so as not to stretch when receiving the weight of a user's foot. In other words, the secondary foot sling 50 may be designed as a stop or limit to the downward movement of a user's foot.

The shoe interface member 60 provides the means for coupling the ankle support apparatus 10 to a shoe (FIG. 1a). In use, the ankle support apparatus 10 may be selectively and releasably coupled to any lace-type shoe as will become apparent below. The shoe interface member 60 includes a proximal end 62 operatively coupled to the side wall 24 of the leg support member 20 adjacent the open bottom 28 thereof. In other words, the shoe interface member 60 may be directly coupled to the sleeve 22 or, in some embodiments, there may be intermediate structures connecting to the two. The shoe interface member 60 extends away from the sleeve 22. Namely, the shoe interface member 60 may extend forwardly and generally perpendicular to the upstanding side wall 24 of the leg support member 20 (FIG. 1a). In one embodiment, the shoe interface member 60 may be pivotally coupled to the sleeve 22 so as to move flexibly when being coupled to a particular configuration of a shoe.

The shoe interface member 60 may include a pair of spaced apart interface sections 64 extending away from the proximal end 62 that define a plurality of apertures 68, the pair of interface sections 64 being generally parallel to one another and define a void therebetween. Each interface section 64 includes a terminal or free end 66 displaced from the proximal end 62 of the shoe interface member 60. The shoe interface member 60 is configured such that the plurality of apertures 68 register or line up with the plurality of lace holes on a lace-up type of shoe, thus enabling a shoe lace to be threaded through respective lace holes and apertures 68 in order to couple the shoe interface member 60 to the shoe. In the same manner that a shoe is tightened using its shoe laces, the compression imparted by the shoe interface member 60 may be tightened by tightening the laces. Tightening the laces may also create an upward force by the primary foot sling 40 on the bottom of a user's foot when positioned in a shoe.

In one embodiment, the shoe interface member 60 includes a tongue tab 70 coupled to the proximal end 62 of the shoe interface member 60. The tongue tab 70 is configured to register or line up with a traditional tongue of a lace-up type shoe. The tongue tab 70 may define a through hole 72 configured to receive a shoe lace (not shown) of the shoe so as to enhance the position of the shoe interface member 60 and its secure attachment to the shoe. It is understood that the tongue tab 70 is situated intermediate the pair of interface sections 64 so that a shoe lace may be selectively inserted through respective apertures 68 and through hole 72.

Another embodiment of the shoe-interfaced ankle support apparatus 10 includes a construction substantially similar to that described previously except as specifically noted below. In this embodiment, the sleeve 22 described previously defines a primary pocket. The sleeve also defines a primary slot in communication with the primary pocket and configured to selectively give access to the primary pocket (FIG. 2a). More particularly, a primary pocket fastener 76 may be moved from a closed configuration preventing access to the primary pocket and an open configuration allowing access to the primary pocket. The primary pocket fastener 76 may be a zipper configured to be selectively moved between open and closed configurations, although the primary pocket fastener 76 may also include a hook and loop combination, snap-fit arrangement, clasp, flap, or the like.

A primary padded panel 78 may be positioned in the primary pocket of the sleeve 22 and can be inserted into or removed from the primary pocket through the primary slot described above (FIG. 3a). The primary padded panel 78 is configured to shield a user's lower leg from impact forces, such as may be inexperienced during recreational activities.

Similarly, the sleeve 22 may define a secondary pocket and a secondary slot in communication with the secondary pocket. Likewise, a secondary pocket fastener 82 may be situated at the secondary slot and is movable between open and closed configuration to selectively provide or restrict access thereto (FIG. 20. A secondary padded panel 84 may also be inserted into or removed from the secondary pocket and, when inserted, enhance protection to a user's lower leg.

In one embodiment, the shoe-interfaced ankle support apparatus 10 includes a heel support member 86 having opposed ends that are operatively coupled to opposed edges, respectively, of the side wall 24 of the leg support member 20 adjacent the open bottom 28 (FIGS. 4a and 4c). The heel support member 86 is configured to extend rearwardly. The heel support member 86 has a generally U-shaped configuration so as to surround the heel of a person's foot when the leg support member 20 is worn about a user's lower leg. The heel support member 86 may be constructed of an elastic material so as to impart compressive force against a user's heel. It is noted that the heel support member 86 is oriented generally perpendicular to the side wall 24 of the leg support member 20. When the leg support member 20 is positioned on a user's lower leg, the primary foot sling 40 cradles a person's foot and the heel support member 86 supports the Achilles tendon of the person's foot.

In another embodiment, the shoe-interfaced ankle apparatus 10 includes an ankle/leg interface 88 having at least one first end releasably coupled to the side wall 24 of the leg support member 20 adjacent the open bottom 28 and at least one second end operatively coupled to the primary foot sling 40 and to the shoe interface member 60. The heel support member 86 may also be operatively coupled to the ankle/leg interface 88. In one embodiment, the ankle/leg interface 88 may have opposed first ends coupled to opposed points of the side wall 24 and opposed second ends coupled to the primary foot sling 40 and to the shoe interface member 60 as shown in FIG. 3a. The ankle/leg interface 88 is essentially a linkage between the leg support member 20 (“an upper portion”) and the other components of the apparatus 10 (“a lower portion”). Accordingly, the leg support member 20 may be released from the other components and either portion may be utilized without the other. (FIG. 4c).

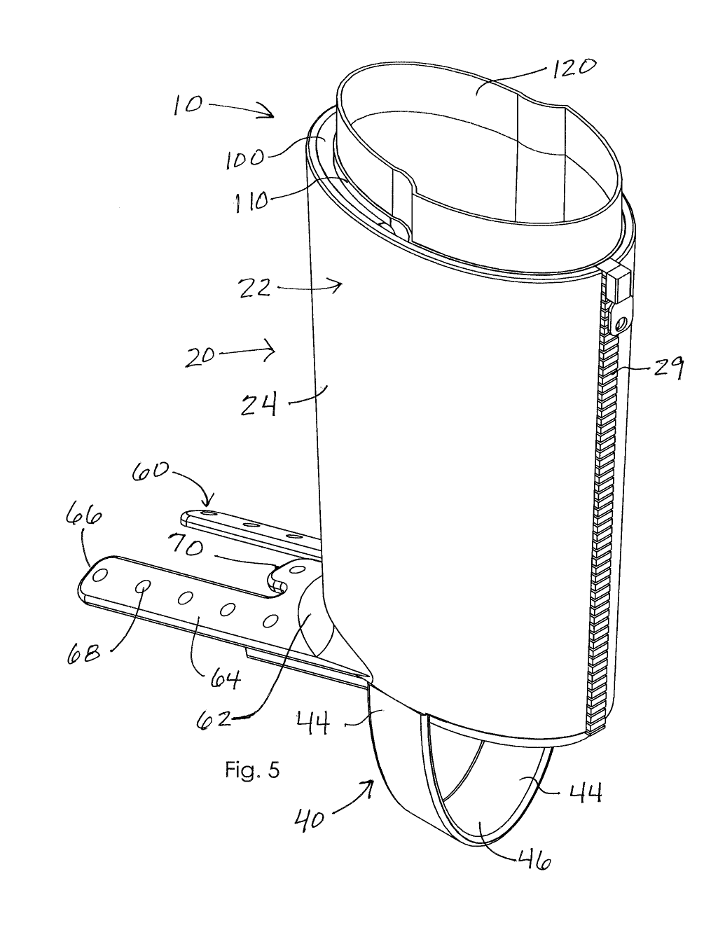

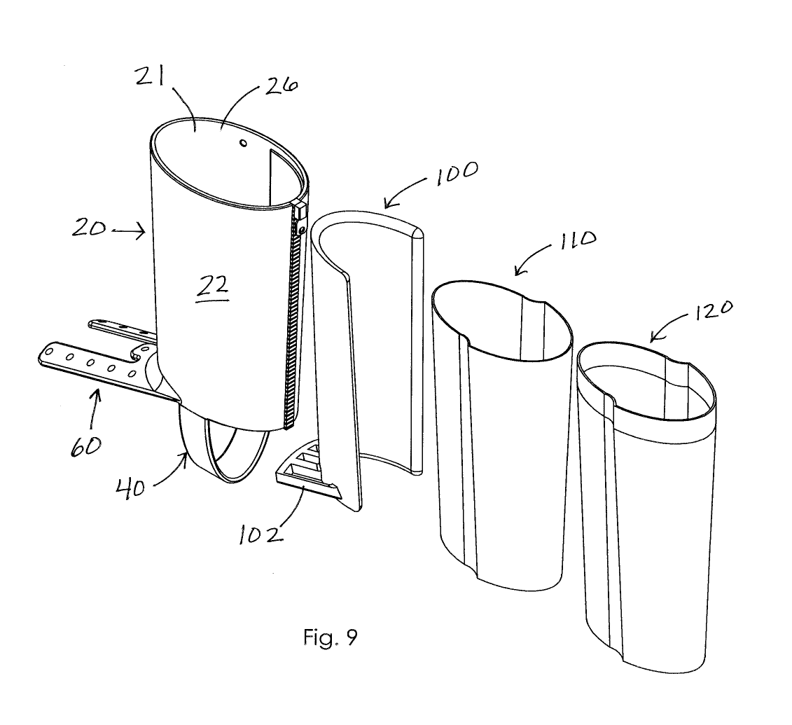

Other aspects of the shoe-interfaced ankle support apparatus 10 are specifically illustrated in FIGS. 5 to 19b of the accompanying drawings. The shoe-interfaced ankle support apparatus 10 includes an inflatable air bladder 100 that, when inflated, applies increased pressure or compression on the ankle of a user. More particularly, the air bladder 100 may have a substantially U-shaped configuration coupled to an inner surface of the leg support member 20 and extending substantially between an open top and open bottom thereof (FIG. 9). The air bladder 100 may also include a front portion 102 that extends forwardly of the open bottom of the leg support member 20 so as to extend beneath a portion of the shoe interface member 60 (FIGS. 4 and 11).

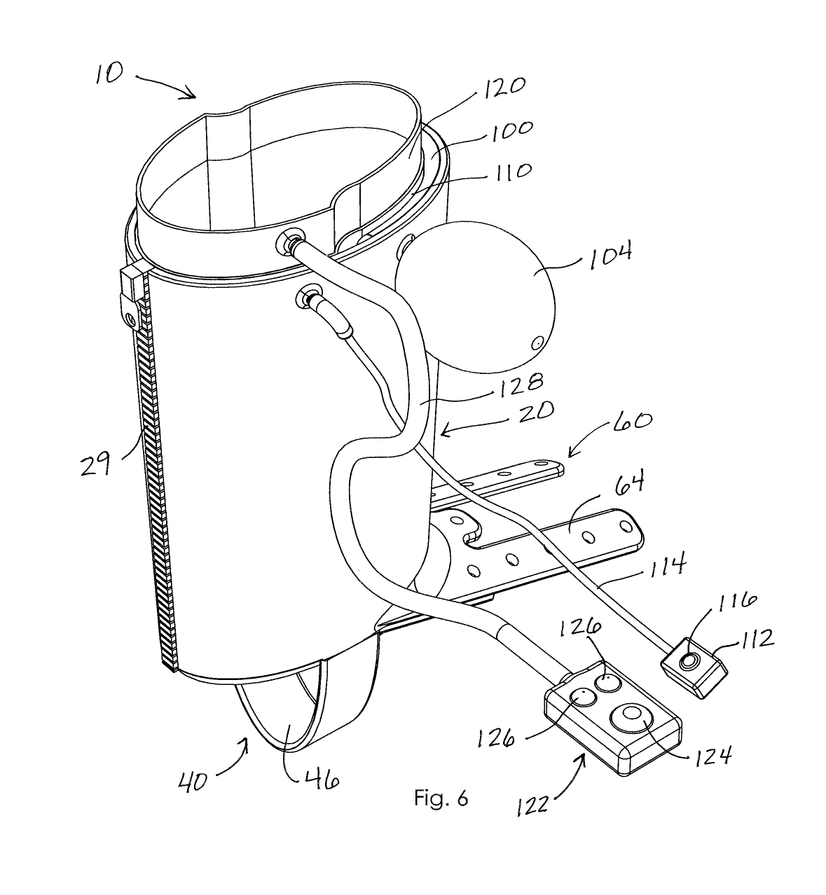

The air bladder 100 may define an open interior area that has an airtight seal so that air may be selectively pumped into or released from the interior area. Specifically, the support apparatus 10 may include a manual air pump 104 in fluid (air) communication with the air bladder 100, the air pump 104 being configured to force a predetermined quantity of ambient air into the air bladder 100 with each squeeze of the air pump 104 (FIG. 6). The air pump 104 may be operatively coupled to the air bladder 100 via an air tube 106 that may pass through the wall of the leg support member 20. In use, a user may connect the air pump 104 to the air bladder 100 if additional compressive pressure on an ankle or lower leg is desired. Although a manual air pump 104 is illustrated in the figures, it is understood that an electric, battery, or hydraulic pump may also be utilized to inflate the air bladder 100.

A heat sleeve 110 is operatively coupled to the leg support member 20 and extends substantially between the open top and open bottom thereof. More particularly, the heat sleeve 110 may include one or more heating elements, such as wires, in operative communication with the leg support member 20 when energized. The heat sleeve 110 may be electrically connected to a battery (not shown) and configured to be selectively energized when heat is desired to be imparted upon the ankle and lower leg of a user wearing the leg support member 20. The heat sleeve 110 may be positioned to the interior of the air bladder 100. The heat sleeve 110 may be displaced from the air bladder 100 so that the air bladder 100 may be inflated which will increase the volume thereof.

A heat sleeve controller 112 is electrically connected to the heat sleeve 110 with a water resistant cord 114. The heat sleeve controller 112 may include a single on/off switch 116 or, in some embodiments, a temperature control dial (not shown). The heat sleeve 110 may also be coupled to a battery (not shown) with the on/off switch 116 being configured to selectively energize the heat sleeve 110 to produce heat.

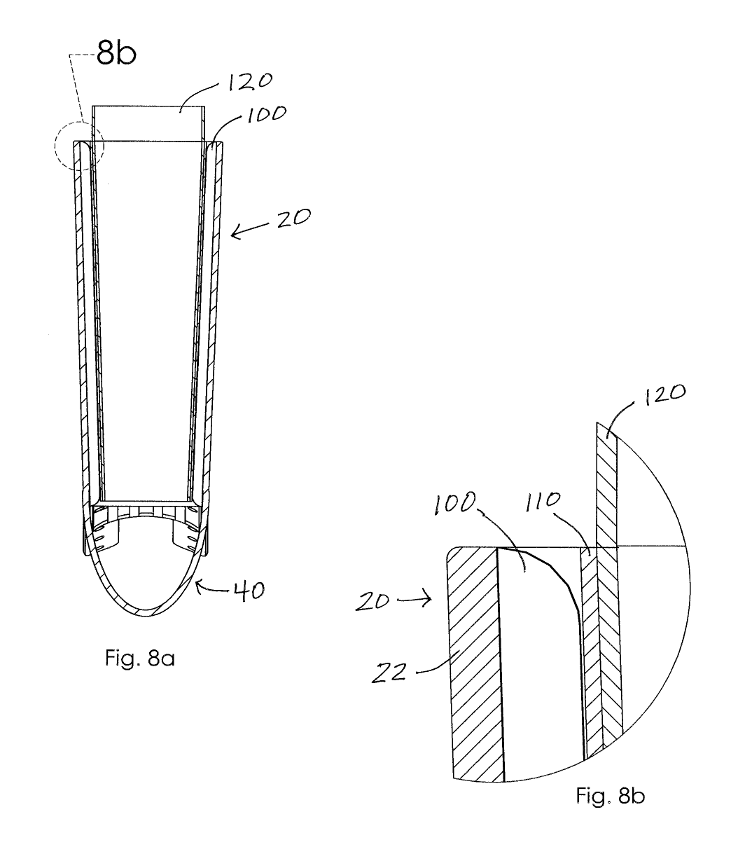

In addition, a transcutaneous electrical nerve stimulation (“TENS”) sleeve 120 is operatively coupled to the leg support member 20 and extends substantially between the open top and open bottom thereof. Specifically, the TENS sleeve 120 includes a primary body portion having a plurality of electrical nerve stimulator members situated in or on the primary body portion. Preferably, the TENS sleeve 120 is positioned interiorly adjacent the heat sleeve 110 (FIG. 8b).

A TENS sleeve controller 122 is electrically connected to the TENS sleeve 120 with a a data cable 128. The TENS sleeve controller 122 may include an activation switch 116 and input buttons 126 configured to increase or decrease electrical stimulation. The TENS sleeve controller 122 enables the TENS sleeve 120 to be operated from a position displaced from the leg support member 20.

Electrical stimulation imparted by the TENS sleeve reduces pain through nociceptive (pain arising from the stimulation of nerve cells) inhibition at the presynaptic level in the dorsal horn, thus limiting its central transmission. The electrical stimuli on the skin preferentially activate low-threshold, myelinated nerve fibers. The afferent input from these fibers inhibits propagation of nociception carried in the small, unmyelinated C fibers by blocking transmission along these fibers to the target or T cells located in the substantia gelatinosa (laminae 2 and 3) of the dorsal horn.

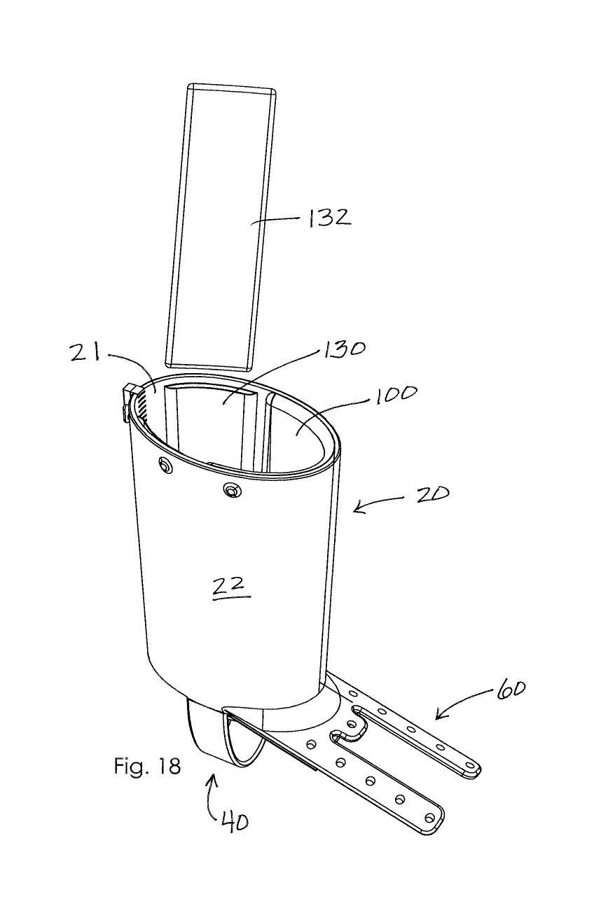

The inner surface 21 of the leg support member 20 includes a pouch 130 configured to receive a hot/cold pack 132 (FIG. 18). More particularly, the pouch 130 may extend substantially between the open top of the leg support member 20 and the open bottom thereof. The pouch defines an interior space and an open upper end that allows access to the interior space. In an embodiment, the leg support member 20 includes a pair of opposed pouches 130 so that heat or cold temperature may be selectively imparted to the walls of the leg support member 20 and, as a result, to the leg of a user wearing the leg support member 20.

In use, a person having a sprained ankle first puts on the shoe-interfaced ankle support apparatus 10 by pulling the leg support member 20 over his foot and onto the lower (“calf”) portion of his leg. In some embodiments, the leg support member 20 may be loosened by releasing or disengaging the sleeve fastener 29. The leg support member 20 should be oriented on the user's leg such that the shoe interface member 60 extends forwardly. The leg support member 20 should be pulled upwardly on the user's leg until the person's foot is cradled by the primary foot sling 40 and, in some embodiments, engaged by the heel support member 86. Then, the user may insert his foot, including the primary foot sling 40, into a shoe of the lace-up type such that the shoe interface member 60 rests upon the shoe lace portion of the shoe. The shoe laces of the shoe may then be restrung to couple the interface sections to the shoe lace portion as described above. The leg support member 20 may be tightened as described above. Further, the additional features of the leg support member 20, such as the inflatable air bladder 100, heat sleeve 110, and TENS sleeve 120 may be actuated as described above to provide additional support and therapy to the lower leg of a user.

It is understood that while certain forms of this invention have been illustrated and described, it is not limited thereto except insofar as such limitations are included in the following claims and allowable functional equivalents thereof.

1. A shoe-interfaced ankle support apparatus for wear on a lower leg and foot of a user while wearing a shoe having a sole, an upper portion, and a lace portion defining a plurality of holes configured to receive a shoe lace, said shoe-interfaced ankle support apparatus, comprising:
a leg support member having a continuous side wall defining an open top and open bottom configured to be worn on the user's lower leg;
an air bladder in operative communication with an inner surface of said side wall of said leg support member, said air bladder movable between an inflated configuration and a deflated configuration; and
a shoe interface member having a proximal end operatively coupled to said side wall of said leg support member adjacent said open bottom and extending away from said side wall, said shoe interface member having a plurality of spaced apart apertures configured to register with respective holes of the lace portion of the shoe and to receive the shoelace of the shoe therethrough.

2. The shoe-interfaced ankle support apparatus as in claim 1, further comprising a primary foot sling having opposed first and second ends coupled to said side wall of said leg support portion adjacent said closed bottom, said foot sling having a cradle portion configured to extend loosely across said open bottom of said leg support portion so as to selectively receive and cradle the foot of the user.

3. The shoe-interfaced ankle support apparatus as in claim 1, further comprising an air pump in fluid communication with said air bladder, said air pump configured to selectively move said air bladder to said inflated configuration upon a squeezing action asserted on said air pump.

4. The shoe-interfaced ankle support apparatus as in claim 1, comprising a transcutaneous electrical nerve stimulation (“TENS”) sleeve operatively coupled to said inner surface of said leg support member, said TENS sleeve configured to therapeutically stimulate nerves in a user's leg when wearing said leg support member and when energized.

5. The shoe-interfaced ankle support apparatus as in claim 4, comprising a heat sleeve intermediate said air bladder and said TENS sleeve and in operative communication with said leg support member, said heat sleeve having a heating element situated internally and configured to warm said leg support member when energized.

6. The shoe-interfaced ankle support apparatus as in claim 1, comprising a heat sleeve operatively coupled to said inner surface of said leg support member, said heat sleeve having a heating element situated internally and configured to warm said leg support member when energized.

7. The shoe-interfaced ankle support apparatus as in claim 5, wherein said inner surface of said leg support member includes a pouch defining an open top in communication with an interior area, said pouch being configured to receive one of a heating pad or a cooling pad.

8. The shoe-interfaced ankle support apparatus as in claim 1, wherein said inner surface of said leg support member includes a pouch defining an open top in communication with an interior area, said pouch being configured to receive one of a heating pad or a cooling pad.

9. The shoe-interfaced ankle support apparatus as in claim 5, wherein:
said primary foot sling is operatively coupled to said shoe interface member;
said primary foot sling is constructed of an elastic material;
said leg support member includes a sleeve configured to surround the lower leg of the user and is constructed of an elastic material that provides compression on the lower leg.

10. The shoe-interfaced ankle support apparatus as in claim 1, wherein said shoe interface member includes a pair of interface sections extending away from said proximal end and defining said plurality of apertures, said pair of interface sections being generally parallel to one another and defining a void therebetween.

11. The shoe-interfaced ankle support apparatus as in claim 1, wherein said shoe interface member includes a tongue tab coupled to said proximal end thereof, said tongue tab being positioned to register with a tongue of the shoe and defining a through hole configured to receive the shoe lace therethrough and enhance the selective coupling of the shoe interface member to the shoe.

12. A shoe-interfaced ankle support apparatus for wear on a lower leg and foot of a user while wearing a shoe having a sole, an upper portion, and a lace portion defining a plurality of holes configured to receive a shoe lace, said shoe-interfaced ankle support apparatus, comprising:
a leg support member having a continuous side wall defining an open top and open bottom configured to be worn on the user's lower leg;
an air bladder in operative communication with an inner surface of said side wall of said leg support member, said air bladder movable between an inflated configuration and a deflated configuration;
a heat sleeve operatively coupled to said inner surface of said leg support member, said heat sleeve having a heating element situated internally and configured to warm said leg support member when energized; and
a shoe interface member having a proximal end operatively coupled to said side wall of said leg support member adjacent said open bottom and extending away from said side wall, said shoe interface member having a plurality of spaced apart apertures configured to register with respective holes of the lace portion of the shoe and to receive the shoelace of the shoe therethrough.

13. The shoe-interfaced ankle support apparatus as in claim 12, further comprising a transcutaneous electrical nerve stimulation (“TENS”) sleeve operatively coupled to said inner surface of said leg support member, said TENS sleeve configured to therapeutically stimulate nerves in a user's leg when wearing said leg support member and when energized.

14. The shoe-interfaced ankle support apparatus as in claim 13, wherein said inner surface of said leg support member includes a pouch defining an open top in communication with an interior area, said pouch being configured to receive one of a heating pad or a cooling pad.

15. The shoe-interfaced ankle support apparatus as in claim 12, wherein said inner surface of said leg support member includes a pouch defining an open top in communication with an interior area, said pouch being configured to receive one of a heating pad or a cooling pad.

16. The shoe-interfaced ankle support apparatus as in claim 12, further comprising an air pump in fluid communication with said air bladder, said air pump configured to selectively move said air bladder to said inflated configuration upon a squeezing action asserted on said air pump.

17. The shoe-interfaced ankle support apparatus as in claim 12, further comprising a primary foot sling having opposed first and second ends coupled to said side wall of said leg support portion adjacent said closed bottom, said foot sling having a cradle portion configured to extend loosely across said open bottom of said leg support portion so as to selectively receive and cradle the foot of the user.

18. The shoe-interfaced ankle support apparatus as in claim 12, wherein:
said primary foot sling is operatively coupled to said shoe interface member;
said primary foot sling is constructed of an elastic material;
said leg support member includes a sleeve configured to surround the lower leg of the user and is constructed of an elastic material that provides compression on the lower leg.

19. The shoe-interfaced ankle support apparatus as in claim 12, wherein said shoe interface member includes a pair of interface sections extending away from said proximal end and defining said plurality of apertures, said pair of interface sections being generally parallel to one another and defining a void therebetween.

20. The shoe-interfaced ankle support apparatus as in claim 12, wherein said shoe interface member includes a tongue tab coupled to said proximal end thereof, said tongue tab being positioned to register with a tongue of the shoe and defining a through hole configured to receive the shoe lace therethrough and enhance the selective coupling of the shoe interface member to the shoe.

21. The shoe-interfaced ankle support apparatus as in claim 13, wherein said TENS sleeve includes a remote control configured to operate said TENS sleeve from a location displaced from said leg support member.
